(19) United States
(12) Patent Application Publication
(10) Pub. No.: US 2004/0033954 A1
(43) Pub. Date: Feb. 19, 2004

(54) Therapeutic method for combined use of modified CNTF and thiadolidinedione

(76) Inventor: Mark W. Sleeman, Mahopac, NY (US)

Correspondence Address: REGENERON PHARMACEUTICALS, INC, 777 OLD SAW MILL RIVER ROAD, TARRYTOWN, NY, 10591, US

(21) Appl. No.: 10/639,107

(22) Filed: Aug. 12, 2003

Related U.S. Application Data
(60) Provisional application No. 60/403,751, filed on Aug. 15, 2002.

(51) Int. Cl. A61K 38/18; A61K 31/4439; A61K 31/426

(52) U.S. Cl. 514/12; 514/342; 514/369

(57) ABSTRACT

A combined therapy for the treatment of non-insulin dependent diabetes mellitus (NIDDM) with ciliary neurotrophic factor (CNTF) or a modified CNTF, and a thiazolidinedione.

### Statement of Related Applications

This application claims priority under 35 USC § 119(e) to provisional application U.S. Ser. No. 60/403,751 filed Aug. 15, 2002, which application is herein specifically incorporated by reference in its entirety.

### Reference to Sequence Listing

This application includes a Sequence Listing hereinto attached, which is considered to be part of the disclosure of the invention.

### BACKGROUND OF THE INVENTION

1. Field of the Invention

The present invention relates to therapeutic CNTF-related polypeptides useful for the treatment of neurological or other diseases or disorders.

2. Statement of Related Art

Ciliary neurotrophic factor (CNTF) is a protein that is required for the survival of embryonic chick ciliary ganglion neurons in vitro (Manthorpe et al. (1980) J. Neurochem. 34:69-75). CNTF variants having specific physical, biochemical and pharmacological properties are known. For example, Axokine™ (SEQ ID NO:1) is a modified CNTF molecule with improved physical and chemical properties, which retains the ability to interact with and activate the CNTF receptor. (Panayotatos et al. (1993) J. Biol. Chem. 268: 19000-19003).

Rosiglitazone maleate is an antidiabetic agent which acts primarily by increasing insulin sensitivity. It is a member of the thiazolidinedione class of compounds, also called glitazones (Lebovitz (2002) Diab/Metab Res and Revs 18(S2): S23-S29) and is structurally related to troglitazone and pioglitazone. It is used in the management of non-insulin dependent diabetes mellitus (NIDDM). Rosiglitazone maleate improves glycemic control while reducing circulating insulin levels. Pharmacological studies in animal models indicate that it improves sensitivity to insulin in muscle and adipose tissue and inhibits hepatic gluconeogenesis. Rosiglitazone is commercially available as Avandia™.

### SUMMARY OF THE INVENTION

In a first aspect, the invention features a method of treating non-insulin dependent diabetes mellitus (NIDDM) comprising administering to a patient in need thereof a composition comprising a ciliary neurotrophic factor (CNTF), or a modified CNTF and one or more thiazolidinediones. In one embodiment, the modified CNTF is Axokine™ (SEQ ID NO: 1) and the thiazolidinedione are one or both of rosiglitazone and rosiglitazone maleate.

In a second aspect, the invention features a method of preventing or decreasing weight gain, hepatotoxicity and other detrimental side effects to the liver that may be exacerbated by the administration of thiazolidinediones, comprising administering a CNTF or modified CNTF in conjunction with, prior to or subsequent to the administration of the thiazolidinediones. In a preferred embodiment such thiazolidinediones comprise rosiglitazone and/or rosiglitazone maleate. One commercially available form of rosiglitazone is Avandia™ (Smith-Kline Beecham). In another preferred embodiment, the modified CNTF is Axokine™ (SEQ ID NO: 1)(Regeneron Pharmaceuticals, Inc).

Other objects and advantages will become apparent from a review of the ensuing detailed description.

### BRIEF DESCRIPTION OF THE FIGURES

FIG. 1 is a graph of the effect of co-administration of Axokine™ (SEQ ID NO:1) (0.1 or 0.3 mg/kg s.c.), Avandia™ (3 or 10 mg/kg/day oral gavage) , or vehicle for 12 days on body weight in C57BL/KsJ db/db mice (% change from the first day of treatment). At day 5 (arrow), two combined treatment groups were initiated receiving Avandia™ (3 mg/kg/day, oral gavage)+Axokine™ (0.1 or 0.3 mg/kg s.c.). All data represents the mean (n≧6)±SEM.

FIGS. 2A-B is a graph of liver (A) and adipose tissue (B) weight from C57BL/KsJ db/db mice treated as described in Example 2.

FIG. 3 is a graph showing the effect of Axokine™ (SEQ ID NO: 1)and/or Avandia™ treatment on serum glucose in C57BL/KsJ db/db mice treated as described in Example 3. Each point represents the mean±SEM (N=6).

FIG. 4 is a graph showing the effect of Axokine™ (SEQ ID NO: 1)and/or Avandia™ treatment on serum triglyceride in C57BL/KsJ db/db mice treated as described in Example 3. Each point represents the mean±SEM (N=6).

FIG. 5 is a graph showing the effect of Axokine™ (SEQ ID NO: 1) and/or Avandia™ treatment on serum cholesterol in C57BL/KsJ db/db mice treated as described in Example 3. Each point represents the mean±SEM (N=6).

FIG. 6 is a graph showing the effect of Axokine™ (SEQ ID NO: 1) and/or Avandia™ treatment on the ratio of the liver associated enzymes, alanine aminotransferase (ALT) and aspartate aminotransferase (AST) in C57BL/KsJ db/db mice treated as described in Example 3. Each point represents the mean±SEM (N=6).

### DETAILED DESCRIPTION OF THE INVENTION

Before the present methods are described, it is to be understood that this invention is not limited to particular methods, and experimental conditions described, as such methods and conditions may vary. It is also to be understood that the terminology used herein is for the purpose of describing particular embodiments only, and is not intended to be limiting, since the scope of the present invention will be limited only by the appended claims.

As used in this specification and the appended claims, the singular forms “a”, “an”, and “the” include plural references unless the context clearly dictates otherwise. Thus for example, references to “a method” include one or more methods, and/or steps of the type described herein and/or which will become apparent to those persons skilled in the art upon reading this disclosure and so forth.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All publications mentioned herein are incorporated herein by reference to describe the methods and/or materials in connection with which the publications are cited.

General Description

The thiazolidinediones are a new class of drugs for the treatment of patients with Type 2 diabetes (NIDDM). These drugs improve insulin sensitivity, primarily in fat and muscle, and reduce endogenous insulin levels. However the finding of severe hepatotoxicity, sometimes fatal, has offset the potential advantages and the first generation of compounds (e.g. troglitazone) were removed from the US market in Mar. 2000. Two newer thiazolidinediones, rosiglitazone (Avandia, Smith Kline Beecham, Philadelphia, Pa., U.S.A.) and pioglitazone (Actos, Takeda, Ill., U.S.A. and Lilly, Indianapolis, Ind., U.S.A.) have been introduced however the risks for hepatotxicity have not been fully assessed. 100191 We have previously found that Axokine T (SEQ ID NO:1) has a profound effect on food intake and body weight as well as on blood glucose and tolerance and serum insulin levels. We have now for the first time shown that co-administration with one member of the thiazolidinediones class of compounds may be beneficial to overcome these side effects for the treatment of NIDDM.

CNTF and CNTF Variants

The method of the invention encompasses the combined administration of CNTF, or a modified variant thereof, and a thiazolidinedione. In a preferred embodiment, the method of the invention is practiced with CNTF or a modified CNTF. A modified CNTF molecule termed Axokine™ (SEQ ID NO:1) (Regeneron Pharmaceuticals, Inc.) described in U.S. Pat. No.6,472,178, the disclosure of which is herein specifically incorporated by reference. Modified CNTF molecules useful in the method of the present invention include, for example, RG297, RG242, rHCNTF, rHCNTFΔ13, Ax-1, Ax-13, Ax-15, or any other modified CNTF that enhances the molecules therapeutic properties.

The CNTF and modified CNTF molecules useful for practicing the present invention may be prepared by cloning and expression in a prokaryotic or eukaryotic expression system as described, for example in Masiakowski et al. (1991) J. Neurosci. 57:1003-1012 and in WO 91/04316. The recombinant neurotrophin gene may be expressed and purified utilizing any number of methods.

Thiazolidinediones and Related Molecules

The thiazolidinediones (TZD) class of compounds, such as pioglitazone, rosiglitazone, and troglitazone, are anti-diabetic agents believed to act by activation of peroxisome proliferator-activated receptors-gamma (PPAR-γ). PPAR-γ is highly expressed in white adipose tissue with lower levels in skeletal muscle and liver. These drugs improve insulin sensitivity, and reduce endogenous insulin levels. Unfortunately, these compounds are associated with sometimes fatal hepatotoxicity, resulting in removal of troglitazone from the U.S. market in 2000. The two newer compounds, rosiglitazone and pioglitazone have been introduced and appear to have decreased hepatotoxicity relative to troglitazone. TZD class compounds have side effects of edema, slight reductions in hemoglobin and hematocrit, weight gain, and alterations in plasma lipid profiles.

Methods of Administration

The invention provides methods of treatment comprising administering to a subject an effective amount of a pharmaceutical composition(s) comprising CNTF or modified CNTF and one or more thiazolidinediones. The active components used in the method of the invention may be administered separately, simultaneously, sequentially, or together. Various delivery systems are known and can be used to administer the composition of the invention, e.g., encapsulation in liposomes, microparticles, microcapsules, recombinant cells capable of expressing the compound, receptor-mediated endocytosis (see, e.g., Wu and Wu, 1987, J. Biol. Chem. 262:4429-4432), construction of a nucleic acid as part of a retroviral or other vector, etc. Methods of introduction can be enteral or parenteral and include but are not limited to intradermal, intramuscular, intraperitoneal, intravenous, subcutaneous, intranasal, intraocular, and oral routes. The compounds may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local. Administration can be acute or chronic (e.g. daily, weekly, monthly, etc.) or in combination with other agents. Pulmonary administration can also be employed, e.g., by use of an inhaler or nebulizer, and formulation with an aerosolizing agent.

In another embodiment, the active agent can be delivered in a vesicle, in particular a liposome, in a controlled release system, or in a pump. In another embodiment where the active agent of the invention is a nucleic acid encoding a protein, the nucleic acid can be administered in vivo to promote expression of its encoded protein, by constructing it as part of an appropriate nucleic acid expression vector and administering it so that it becomes intracellular, e.g., by use of a retroviral vector (see, for example, U.S. Pat. No. 4,980,286), by direct injection, or by use of microparticle bombardment, or coating with lipids or cell-surface receptors or transfecting agents, or by administering it in linkage to a homeobox-like peptide which is known to enter the nucleus (see e.g., Joliot et al., 1991, Proc. Natl. Acad. Sci. USA 88:1864-1868), etc. Alternatively, a nucleic acid can be introduced intracellularly and incorporated within host cell DNA for expression, by homologous recombination.

A composition useful in practicing the methods of the invention may be a liquid comprising an agent of the invention in solution, in suspension, or both. The term “solution/suspension” refers to a liquid composition where a first portion of the active agent is present in solution and a second portion of the active agent is present in particulate form, in suspension in a liquid matrix. A liquid composition also includes a gel. The liquid composition may be aqueous or in the form of an ointment.

An aqueous suspension or solution/suspension useful for practicing the methods of the invention may contain one or more polymers as suspending agents. Useful polymers include water-soluble polymers such as cellulosic polymers and water-insoluble polymers such as cross-linked carboxyl-containing polymers. An aqueous suspension or solution/suspension of the present invention is preferably viscous or muco-adhesive, or even more preferably, both viscous or mucoadhesive.

Pharmaceutical Compositions

In one embodiment, the invention provides a pharmaceutical composition comprising CNTF or a modified CNTF and, together or separately, a composition comprising one or more thiazolidinediones, and a pharmaceutically acceptable carrier. The term “pharmaceutically acceptable” means approved by a regulatory agency of the Federal or a state government or listed in the U.S. Pharmacopeia or other generally recognized pharmacopeia for use in animals, and more particularly, in humans. The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. Examples of suitable pharmaceutical carriers are described in “Remington's Pharmaceutical Sciences” by E. W. Martin.

The composition of the invention can be formulated as neutral or salt forms. Pharmaceutically acceptable salts include those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2-ethyl amino ethanol, histidine, procaine, etc.

The amount of the composition of the invention that will be effective for its intended therapeutic use can be determined by standard clinical techniques based on the present description. In addition, in vitro assays may optionally be employed to help identify optimal dosage ranges. Generally, suitable dosage ranges for intravenous administration are generally about 20-500 micrograms of active compound per kilogram body weight. Suitable dosage ranges for intranasal administration are generally about 0.01 pg/kg body weight to 1 mg/kg body weight. Effective doses may be extrapolated from dose-response curves derived from in vitro or animal model test systems.

For systemic administration, a therapeutically effective dose can be estimated initially from in vitro assays. For example, a dose can be formulated in animal models to achieve a circulating concentration range that includes the IC₅₀ as determined in cell culture. Such information can be used to more accurately determine useful doses in humans. Initial dosages can also be estimated from in vivo data, e.g., animal models, using techniques that are well known in the art. One having ordinary skill in the art could readily optimize administration to humans based on animal data.

Dosage amount and interval may be adjusted individually to provide plasma levels of the compounds that are sufficient to maintain therapeutic effect. In cases of local administration or selective uptake, the effective local concentration of the compounds may not be related to plasma concentration. One having skill in the art will be able to optimize therapeutically effective local dosages without undue experimentation.

The amount of compound administered will, of course, be dependent on the subject being treated, on the subject's weight, the severity of the affliction, the manner of administration, and the judgment of the prescribing physician. The therapy may be repeated intermittently while symptoms are detectable or even when they are not detectable. The therapy may be provided alone or in combination with other drugs.

Specific Embodiments

As shown below, the administration of Axokine™ (SEQ ID NO:1) results in decreased side effects associated with rosiglitazone treatment. The effect of daily administration of Axokine™ ±Avandia™ on body weight and side effects associated with rosiglitazone treatment is described below. The results show that co-administration of Axokine™ with rosiglitazone significantly attenuated elevated serum parameters of glucose, triglyerides, creatinine, ALT/AST ratio, BUN, and insulin, associated with administration of rosiglitazone alone.

It had been previously demonstrated that administration of rosiglitazone resulted in increased body weight (Chaput et al. (2000) Biochem. Biophys. Res. Commun. 271:445-450) and that administration of certain thiazolidinediones such as troglitazone have been associated with idiosyncratic hepatic reaction leading to hepatic failure and death in some patients (Lebovitz et al. (2002) Diabetes Care 25(5) 815-21). The combined use of Axokine™ and and anti-diabetic thiazolidinedione can thus be useful for achieving the desired therapeutic effect without the side effects associated with thiazolidinediones.

### EXAMPLES

The following example is put forth so as to provide those of ordinary skill in the art with a complete disclosure and description of how to make and use the methods and compositions of the invention, and are not intended to limit the scope of what the inventors regard as their invention. Efforts have been made to ensure accuracy with respect to numbers used (e.g., amounts, temperature, etc.) but some experimental errors and deviations should be accounted for. Unless indicated otherwise, parts are parts by weight, molecular weight is average molecular weight, temperature is in degrees Centigrade, and pressure is at or near atmospheric.

### Example 1

### Co-administration of Axokine™ and Avandia™.

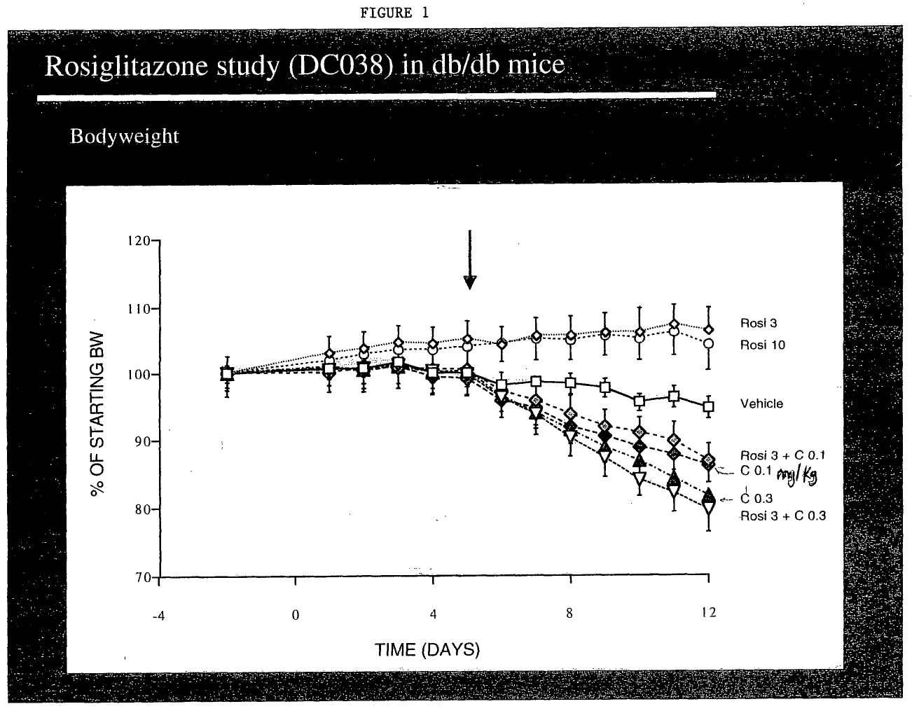

C57BL/KsJ db/db mice were treated by daily administration of Axokine™ (SEQ ID NO:1) (0.1 or 0.3 mg/kg s.c.), Avandia™ (3 or 10 mg/kg/day oral gavage), and vehicle (vehicle) for 10 days, and the effect on body weight determined (FIG. 1, shown as the % change from the first day of treatment). Daily injection Axokine™ caused weight loss in db/db mice and this was associated with decreased food intake. Increasing doses of Axokine™ resulted in a further reduction in body weight from vehicle control groups (ANOVA, P<0.0001 in all cases). A combined treatment group consisted of Avandia™ (3 mg/kg/day, oral gavage) +Axokine™ (0.1 or 0.3 mg/kg s.c.) administered for the last 10 days (arrow, FIG. 1). All data represents the mean (n>6)±SEM.

### Example 2

### Effect of Axokine™ and Avandia™ on liver and adipose tissue weight.

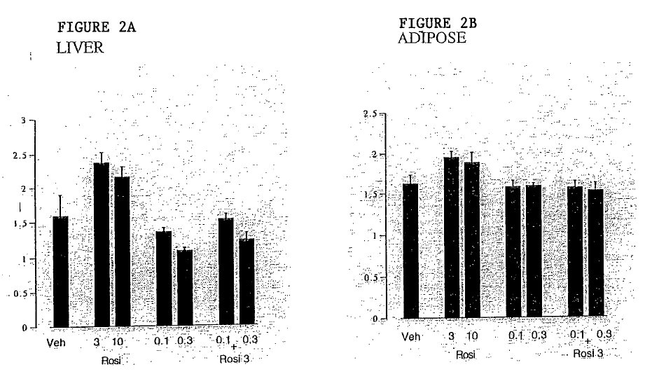

week old male db/db mice were injected daily with vehicle, Axokine™ (SEQ ID NO:1) (0.1 and 0.3 mg/kg/day) and/or Avandia™ (3 or 10 mg/kg/day oral gavage). 24 hours after completion of the study animals were sacrificed and the total liver (FIG. 2A) and epididymal adipose mass (FIG. 2B) were removed and weighed The results are shown in FIG. 2. The db/db mice normally exhibit a mild hepatomegaly, and for those used in these experiments this is depicted by an increased liver weight/body weight ratio of 3.5% compared with 2.8% for age and gender matched lean mice (data not shown).

### Example 3

### Effect of Avandia™±Axokine™ on serum parameters.

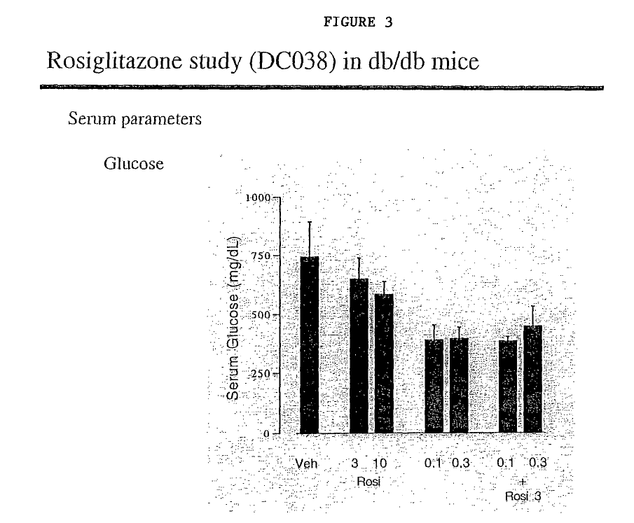

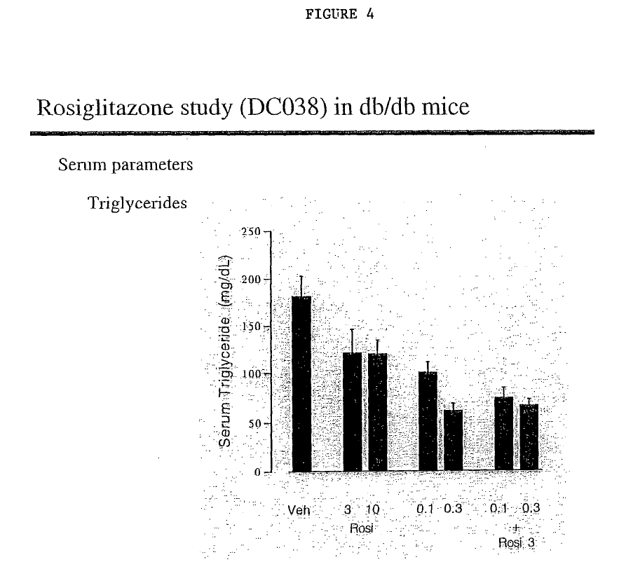

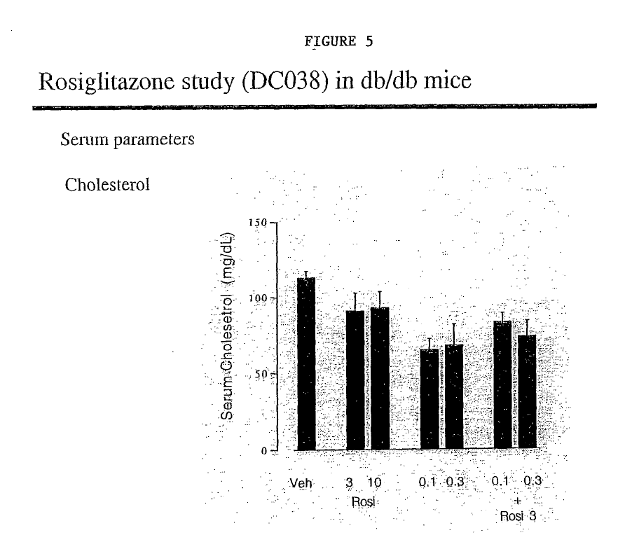

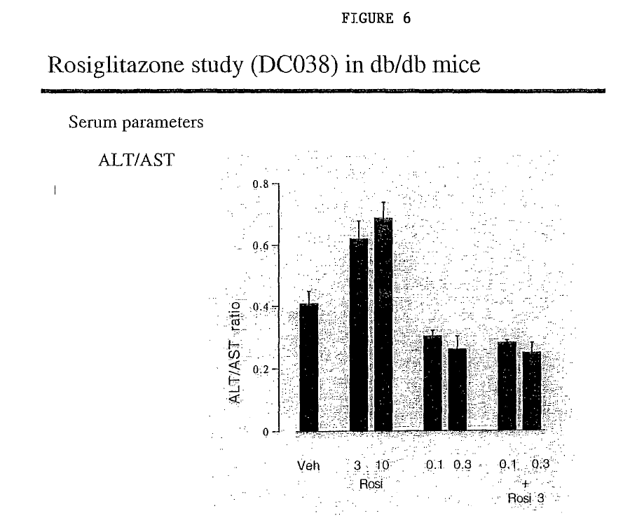

week old male db/db mice were injected daily with vehicle, Axokine™ (0.1 and 0.3 mg/kg/day) and/or Avandia™ (3 or 10 mg/kg/day oral gavage). Serum was taken from animals 24 hours after the last treatment and measured for glucose (FIG. 3), triglyceride (FIG. 4), cholesterol (FIG. 5) and the ratio of the liver associated enzymes, alanine aminotransferase (ALT) and aspartate aminotransferase (AST) (FIG. 6).

### Example 4

### Effect of Avandia™±Axokine™ on fatty liver.

The effects of Avandia™ administration ±Axokine™ (SEQ ID NO:1) on fatty liver of db/db mice was examined. 10-week-old C57Ks/J db/db male mice were treated for 13 days with either vehicle or Avandia™ alone (3 or 10 mg/kg/day) by oral gavage or ±Axokine™ (0.1 or 0.3 mg/kg/day) in the last 8 days. 24 hours after the last administration (day 14) all animals were anesthetized , the liver removed and rapidly frozen until it could be processed. The tissues were sectioned, mounted on glass, and stained with Oil-Red O for the detection of neutral lipid. Control db/db male mice who received the vehicle oral gavage (0.5% carboxymethylcellulose) showed significant deposition of lipid in the liver tissue consistent for this species. Treatment of db/db mice with Avandia™ alone resulted in increased deposition of lipid as shown by increased staining (not shown). The livers of Axokine™ treated db/db mice showed a reduced staining with Oil Red O indicating a reduction in neutral lipid. The combined treatment of Avandia™ and Axokine™ shows a significant attenuation of the result seen with Avandia™ alone.

What is claimed is:

1. A method of treating non-insulin dependent diabetes mellitus (NIDDM), comprising administering to a patient in need thereof a ciliary neurotrophic factor (CNTF), and a thiazolidinedione molecule.

2. The method of claim 1, wherein the CNTF is a modified CNTF.

3. The method of claim 2, wherein the modified CNTF is Axokine™.

4. The method of claim 1, wherein the thiazolidinedione is rosiglitazone or pioglitazone.

5. The method of claim 1, wherein CNTF and rosiglitazone are administered.

6. The method of claim 1, wherein Axokine™ and rosiglitazone are administered.

7. The method of claim 1, wherein administration is subcutaneous, intramuscular, intradermal, intraperitoneal, intravenous, intranasal, epidural, or oral.

8. The method of claim 1, wherein the subject is a human subject.

9. The method of claim 1 wherein a CNTF and a thiazolidinedione are administered sequentially.

10. The method of claim 1 wherein a CNTF and a thiazolidinedione are administered simultaneously.

11. A method of reducing undesirable side effects associated with administration of a thiazolidinedione, comprising co-administering to a patient being treated with a thiazolidinedione, a composition comprising a ciliary neurotrophic factor (CNTF).

12. The method of claim 11, wherein the CNTF is CNTF.

13. The method of claim 11, wherein the CNTF is a modified CNTF.

14. The method of claim 13, wherein the modified CNTF is Axokine™.

15. The method of claim 11, wherein the thiazolidinedione is rosiglitazone or pioglitazone.

16. The method of claim 11, wherein a CNTF and a thiazolidinedione are administered sequentially.

17. The method of claim 11, wherein a CNTF and a thiazolidinedione are administered simultaneously.

18. The method of claim 11, wherein administration is subcutaneous, intramuscular, intradermal, intraperitoneal, intravenous, intranasal, epidural, or oral.

19. The method of claim 11, wherein the subject is a human subject.

20. The method of claim 11, wherein the undesirable side effect is one or more of weight gain and hepatotoxicity.


SEQUENCE LISTING (1 sequence)

SEQ ID NO: 1 - protein, 185 aa, Artificial Sequence
  Description of Artificial Sequencepurified recombinant protein
MAFTEHSPLTPHRRDLASRSIWLARKIRSDLTALTESYVKHQGLNKNINLDSADGMPVASTDRWSELTEAERLQENLQAYRTFHVLLARLLEDQQVHFTPTEGDFHQAIHTLLLQVAAFAYQIEELMILLEYKIPRNEADGMPINVGDGGLFEKKLWGLKVLQELSQWTVRSIHDLRFISSHQTG